(19) United States
(12) Patent Application Publication
(10) Pub. No.: US 2012/0029003 A1
(43) Pub. Date: Feb. 2, 2012

(54) GALECTIN-3 AND STATIN THERAPY

(75) Inventor: Pieter Muntendam, Boxford, MA (US)

(73) Assignee: BG Medicine, Inc., Waltham, MA (US)

(21) Appl. No.: 13/175,523

(22) Filed: Jul. 1, 2011

Related U.S. Application Data
(60) Provisional application No. 61/361,216, filed on Jul. 2, 2010.

(51) Int. Cl. A61K 31/505; A61K 31/366; C12Q 1/26; A61P 9/10; A61P 25/00; A61K 31/40; A61P 9/04

(52) U.S. Cl. 514/275; 514/423; 514/460; 435/25

(57) ABSTRACT

Described herein are materials and methods for predicting and monitoring a heart failure patient's physiological response to treatment with a statin. More specifically, the present invention relates to the endogenous protein galectin-3 and its use as a predictor of response to treatment with 3-hydroxy-3-methylglutaryl coenzyme-A reductase inhibitors, or statins.

### REFERENCE TO RELATED APPLICATIONS

This application claims the benefit of and priority to U.S. Provisional Patent Application No. 61/361,216, filed Jul. 2, 2010, the complete disclosure of which is incorporated by reference herein.

### FIELD OF THE INVENTION

The present invention relates to materials and methods for predicting and monitoring a heart failure patient's physiological response to treatment with a statin. More specifically, the present invention relates to the endogenous protein galectin-3 and its use as a predictor of response to treatment with 3-hydroxy-3-methylglutaryl coenzyme-A reductase inhibitors, or statins.

### BACKGROUND

Congestive heart failure, or heart failure, is a major cause of morbidity and mortality. Approximately 5 million people in the United States suffer from heart failure, with approximately 500,000 new cases diagnosed annually.

Recently the potential beneficial effects of 3-hydroxy-3-methylglutaryl coenzyme-A reductase inhibitors, or statins, have been investigated for the prevention and treatment of heart failure. The non-lipid-lowering or pleiotropic effects of statins have been hypothesized to beneficially modify mechanisms involved in heart failure. Although retrospective post-hoc subgroup analyses of a number of cardiovascular clinical trial datasets as well as small prospective trials appear to provide some preliminary support for the potential beneficial utility of statin therapy in heart failure, two recently published, large scale, prospective randomized trials specifically designed to investigate the benefit of statin therapy in heart failure (the Controlled Rosuvastatin Multinational Trial in Heart Failure (CORONA), and the GISSI-HF Trial) did not demonstrate a significant clinical benefit of statin therapy, specifically rosuvastatin treatment, in heart failure patients. Because of the potential importance of statin therapy in heart failure, there is a need to develop methods to identify those patients with heart failure who are likely to benefit from statin therapy.

### SUMMARY OF THE INVENTION

It has now been discovered that concentrations of the human protein galectin-3 in body fluids can be informative of whether a presenting patient (e.g., human) may benefit from statin treatment as a prophylactic or therapeutic serving to delay or inhibit development of congestive or other type of heart failure. In this manner, statins can be administered to patients who are more likely to benefit from the treatment. Thus, it is envisioned that patients who have experienced heart failure or who have been identified as at risk for heart failure will be tested to measure their circulating galectin-3 levels. The measured galectin-3 levels will permit the identification of two groups of patients: those who may benefit from statin treatment, based on their galectin-3 concentration; and those who are unlikely (or less likely) to benefit. Statins would be administered to those in the first group, whereas other courses of therapy would be selected for those in the second group. In addition, galectin-3 levels can optionally be monitored thereafter as an indicator of disease progression (see, for example, US 2008/0193954) or of therapeutic efficacy.

The present invention therefore provides methods for selecting a therapy for a human, who may be suffering from or at risk of heart failure. The methods include measuring a galectin-3 concentration in a sample, such as a blood sample, a serum sample, or a plasma sample. The measured galectin-3 concentration is indicative of whether the patient is likely to respond favorably to a statin. Favorable responses include, for example, an increased likelihood of survival over a period of time, a reduced progression or development of heart failure, a reduced risk of myocardial infarction, and/or a reduced risk of stroke. Once the therapy is selected, the methods can include repeatedly administering the statin to the patient.

In one embodiment, the method includes measuring a galectin-3 concentration in a body fluid (e.g., blood, serum, or plasma) of a heart failure patient that is a candidate for treatment with a statin compound (e.g. rosuvastatin) for heart failure, prior to such treatment, and comparing the galectin-3 concentration to a galectin-3 concentration observed in other patients treated with the statin compound for heart failure for whom the statin therapeutic agent has proven beneficial.

Similarly, the invention provides a method for assessing a candidate (e.g., a heart failure patient) for treatment with a statin (e.g., rosuvastatin). The method includes measuring a galectin-3 concentration in a body fluid of a patient having a condition treatable with a statin and comparing the measured galectin-3 concentration to a reference galectin-3 concentration. The reference galectin-3 concentration can be derived from observed concentrations of galectin-3 in other patients having the condition, and is indicative of beneficial response in patients having the condition. Statin treatment can be restricted or refused if the measured galectin-3 concentration is different than a reference galectin-3 concentration.

The invention also provides methods for treating a human, who may be suffering from or at risk of heart failure; the methods include repeatedly administering a statin to a patient having a determined galectin-3 blood concentration that is indicative of a survival-enhancing response to the statin. In contrast to the methods of selecting a therapy, which involve the process of measuring a galectin-3 blood concentration, the treatment methods relate to the subsequent administration of the statin. Thus, the treatment methods involve the repeated administration of a statin to a patient whose galectin-3 blood concentration has been determined to be indicative of a survival enhancing response, regardless of how the galectin-3 determination was previously made, or by whom. The administration can be performed by a patient, such as by regularly consuming pills containing the statin according to a prescribed schedule.

The statin is preferably administered in an amount sufficient to inhibit progression or development of heart failure (e.g., congestive heart failure) and is preferably in an amount sufficient to prolong survival (i.e., a survival-enhancing amount) or otherwise enhance the patient's health. For example, the statin may be administered in a myocardial infarction risk-reducing amount and/or in a stroke risk-reducing amount. Exemplary statins include atorvastatin, fluvastatin, lovastatin, mevastatin, pitavastatin, pravastatin, rosuvastatin, and simvastatin. For example, rosuvastatin can be administered, such as at a dose of between 5 and 40 mg/day, or atorvastatin can be administered, such as at a dose of between 10 and 80 mg/day.

In a selected or treated patient, the blood concentration of galectin-3 may be determined to be below a maximum threshold or within a target range defined by a minimum and a maximum threshold. The maximum threshold may be, for example, below 70 ng/ml; below 60 ng/ml; below 40 ng/ml; below 30 ng/mL; below 20 ng/mL; below 15 ng/mL; between 30 and 40 ng/ml; between 25 and 30 ng/ml; between 20 and 25 ng/ml; between 15 and 20 ng/ml; between 10 and 15 ng/ml; between 15 and 25 ng/ml; between 10 and 30 ng/ml; or between 18 and 20 ng/ml.

In certain embodiments, the maximum threshold may be a median concentration in a population. In other embodiments, the maximum threshold may be an average concentration in a population. The maximum threshold may, in some cases, be a concentration observed or exceeded in no more than 50% to 60% of a population; no more than 50% to 70% percent of a population; no more than 50% to 80% percent of a population; no more than 40% to 50% percent of a population; no more than 30% to 50% percent of a population; or no more than 20% to 50%.

The patient's galectin-3 blood concentration can optionally be monitored over the course of the therapy to provide an ongoing indication of disease development or progression and/or of the continued propriety of the course of treatment. For example, the invention permits measurements of changes over time in galectin-3 concentration in a body fluid (e.g., blood, serum, or plasma) of a heart failure patient during the course of treatment with a statin compound for heart failure and comparing the measured change in galectin-3 concentration to changes in galectin-3 concentration observed in other patients treated with the statin compound for heart failure for whom the statin therapeutic agent has proven beneficial.

Monitoring methods can include comparing a galectin-3 concentration during a course of treatment with the statin to an earlier galectin-3 concentration during or prior to the course of treatment. The methods can also include comparing galectin-3 levels measured at several times during the course of treatment to develop a treatment history of galectin-3 concentrations. For example, the invention provides methods for assessing a patient by detecting the presence or absence of an increasing or decreasing galectin-3 concentration in a body fluid (e.g., blood, serum, or plasma) of a heart failure patient being treated with a statin. The presence of an increasing galectin-3 concentration over time is indicative of a worsening congestive heart failure in the patient. The method can include comparing a galectin-3 concentration during a course of treatment with the statin to an earlier galectin-3 concentration during the course of treatment. The method can also include comparing galectin-3 levels at several times during the course of treatment to develop a treatment history of galectin-3 concentrations.

The results of the assessment can be used to inform decisions involving treatment of the patient. For example, if a heart failure patient's galectin-3 concentration is dissimilar to those of other heart failure patients for whom the statin therapeutic agent has proven beneficial, administration of the statin may be not be indicated, and the patient may be offered a different medication or therapeutic option. Alternatively, if the heart failure patient appears to be therapeutically unresponsive to statin treatment, based on the patient's galectin-3 concentration or a change in the patient's galectin-3 concentration, administration of the statin may be discontinued, limited, restricted, otherwise modulated, such as by reducing or increasing the dose or frequency of administration. The patient may be switched to a different medication, such as a different statin or a non-statin therapy.

### DESCRIPTION OF THE DRAWINGS

FIG. 1 depicts galectin-3 blood concentrations in heart failure patients who were being treated with rosuvastatin and who survived or did not survive for one year after the galectin-3 measurement. The horizontal line indicates the median galectin-3 concentration for each population; the “X” marks the average galectin-3 concentration.

FIG. 2 shows a plot of Kaplan-Meier estimates for the primary endpoint by galectin-3 category (i.e., above and at or below median level, 19.0 ng/mL) versus follow-up time.

### DETAILED DESCRIPTION OF THE INVENTION

Applicants have invented a method of predicting and/or monitoring a heart failure patient's physiological response to treatment of heart failure with a 3-hydroxy-3-methylglutaryl coenzyme-A reductase inhibitor (a statin). Statins are widely used for their ability to lower serum cholesterol levels, but have properties beyond cholesterol reduction that may be beneficial for heart failure patients, including improvement in endothelial dysfunction, release of endothelial progenitor cells, anti-inflammatory properties and amelioration of left ventricular remodeling and function in heart failure. However, clinical studies have demonstrated that not every heart failure patient benefits from statin therapy, and have highlighted the need to identify those patients with heart failure who are likely to benefit from statin therapy. Currently-available statins include, in alphabetical order (brand names vary in different regions and countries): atorvastatin (Lipitor, Torvast), cerivastatin (Lipobay, Baycol), fluvastatin (Lescol), lovastatin (Mevacor, Altocor), mevastatin naturally-occurring compound, found in red yeast rice, pitavastatin (Livalo, Pitava), pravastatin (Pravachol, Selektine, Lipostat), rosuvastatin (Crestor), and simvastatin (Zocor, Lipex).

The terms “heart failure,” “HF,” “congestive heart failure,” or “CHF” as used herein, refer to the complex clinical syndrome that impairs the ability of the ventricle to fill with or eject blood. Any structural or functional cardiac disorder can cause HF, with the majority of HF patients having impaired left ventricular (LV) myocardial function. Symptoms of HF include dyspnea (shortness of breath), fatigue, and fluid retention. The American Heart Association (AHA) has identified 4 stages in the development of HF. Patients in stages A and B show clear risk factors but have not yet developed HF. Patients in stages C and D currently exhibit or in the past have exhibited symptoms of HF. For example, Stage A patients are those with risk factors such as coronary artery disease, hypertension or diabetes mellitus who do not show impaired left ventricular (LV) function. Stage B patients are asymptomatic, but have cardiac structural abnormalities or remodeling, such as impaired LV function, hypertrophy or geometric chamber distortion. Stage C patients have cardiac abnormalities and are symptomatic. Stage D patients have refractory HF in which they exhibit symptoms despite maximal medical treatment. They are typically recurrently hospitalized or unable to leave the hospital without specialized intervention.

Galectin-3 is a structurally unique member of a family of multifunctional β-galactoside-binding lectins (Gabius (2006) Crit. Rev. Immunol. 26:43-79). Expression of galectin-3 has been associated with the epithelium and inflammatory cells including macrophages, neutrophils and mast cells. Galectin-3 has been implicated in a variety of biological processes important in heart failure including myofibroblast proliferation, fibrogenesis, tissue repair, cardiac remodeling, and inflammation (Liu et al. (2009) Am. J. Physiol. Heart Circ. Physiol. 296(2):H404-12; Papaspyridonos et al. (2008) Arterioscler. Thromb. Vasc. Biol. 28(3):433-40; Henderson et al. (2006) Proc. Natl. Acad. Sci. USA 103:5060-5065; Sharma et al. (2004) Circulation 110:3121-3128; Sano et al. (2000) J. Immunol. 165(4):2156-64; Kuwabara et al. (1996) J. Immunol. 156(10):3939-44).

Applicants have developed methods permitting the use of circulating galectin-3 protein levels to predict efficacy of statin treatments in heart failure patients. Applicants' research has revealed that in a study of heart failure patients being treated with a statin from whom blood was collected at baseline and who were followed for at least one year, patients who died within the follow-up period had a higher median galectin-3 level than patients who did not die within the follow-up period. Accordingly, knowledge of a patient's galectin-3 level is informative of patient outcome when treated with a statin. Furthermore, as the galectin-3 levels were measured in patients already undergoing statin therapy, galectin-3 levels during a course of therapy are informative of patient outcome, and changes in galectin-3 levels may indicate a changing prognosis. Although higher concentrations of galectin-3 correlate with poor prognosis, Applicants have discovered that even patients with relatively high galectin-3 levels who are treated with statins have a substantial chance of survival at one year, which may indicate that these patients benefit significantly from statin therapy.

In some embodiments, galectin-3 levels in a patient (e.g., a human) treated with a statin may correlate with reduced risk of a primary event (e.g., a cardiovascular event). Thus, in some cases, a patient having galectin-3 levels below a maximum threshold or within a target range defined by a minimum and a maximum threshold may be treated with a statin in an amount sufficient to reduce the risk of a cardiovascular event. In some embodiments, the cardiovascular event may be cardiovascular death. In other embodiments, the cardiovascular event may be myocardial infarction (e.g., fatal or non-fatal). The cardiovascular event may be, in some instances, a stroke (e.g., fatal or non-fatal). In some embodiments, patients treated with a statin and having galectin-3 levels below a maximum threshold or within a target range defined by a minimum and a maximum threshold may have a reduced risk of death independent of the cause of death (e.g., reduced total mortality).

In certain embodiments, the risk of a patient (e.g., a human) dying or suffering a primary event may be reduced in patients administered a statin according to the methods contemplated herein as compared to patients not administered the statin. For example, in some cases, the likelihood of a patient suffering a primary event may be reduced by at least 5%; at least 6%; at least 7%; at least 8%; at least 9%; at least 10%; at least 11%; at least 12%; at least 13%; at least 14%; at least 15%; at least 16%; at least 17%; at least 18%; at least 19%; at least 20%; at least 21%; at least 22%; at least 23%; at least 24%; at least 25%; at least 26%; at least 27%; at least 28%; at least 29%; at least 30%; at least 31%; at least 32%; at least 33%; at least 34%; at least 35%; at least 36%; at least 37%; at least 38%; at least 39%; at least 40%; at least 41%; at least 42%; at least 43%; at least 44%; at least 45%; at least 46%; at least 47%; at least 48%; at least 49%; at least 50%; at least 51%; at least 52%; at least 53%; at least 54%; at least 55%; at least 56%; at least 57%; at least 58%; at least 59%; or at least 60%. In some instances, the risk of a patient suffering a primary event may be reduced by an amount between 5% and 60%; between 5% and 10%; between 10% and 15%; between 15% and 20%; between 20% and 25%; between 25% and 30%; between 30% and 35%; between 35% and 40%; between 40% and 45%; between 45% and 50%; between 50% and 55%; between 55% and 60%; between 10% and 40%; between 15% and 40%, between 20% and 40%; or between 25% and 35%. In some cases, the risk of a patient dying may be reduced by at least 5%; at least 6%; at least 7%; at least 8%; at least 9%; at least 10%; at least 11%; at least 12%; at least 13%; at least 14%; at least 15%; at least 16%; at least 17%; at least 18%; at least 19%; at least 20%; at least 21%; at least 22%; at least 23%; at least 24%; at least 25%; at least 26%; at least 27%; at least 28%; at least 29%; at least 30%; at least 31%; at least 32%; at least 33%; at least 34%; at least 35%; at least 36%; at least 37%; at least 38%; at least 39%; at least 40%; at least 41%; at least 42%; at least 43%; at least 44%; at least 45%; at least 46%; at least 47%; at least 48%; at least 49%; or at least 50%, or be reduced by an amount between 5% and 50%; between 5% and 10%; between 10% and 15%; between 15% and 20%; between 20% and 25%; between 25% and 30%; between 30% and 35%; between 35% and 40%; between 40% and 45%; between 45% and 50%; between 10% and 40%; between 15% and 40%, between 20% and 40%; or between 25% and 35%.

In a selected or treated patient (e.g., a human), the blood concentration of galectin-3 may be determined to be below a maximum threshold or within a target range defined by a minimum and a maximum threshold. The maximum threshold may be, for example, below 70 ng/ml; below 69 ng/ml; below 68 ng/ml; below 67 ng/ml; below 66 ng/ml; below 65 ng/ml; below 64 ng/ml; below 63 ng/ml; below 62 ng/ml; below 61 ng/ml; below 60 ng/ml; below 59 ng/ml; below 58 ng/ml; below 57 ng/ml; below 56 ng/ml; below 55 ng/ml; below 54 ng/ml; below 53 ng/ml; below 52 ng/ml; below 51 ng/ml; below 50 ng/ml; below 49 ng/ml; below 48 ng/ml; below 47 ng/ml; below 46 ng/ml; below 45 ng/ml; below 44 ng/ml; below 43 ng/ml; below 42 ng/ml; below 41 ng/ml; below 40 ng/ml; below 39 ng/ml; below 38 ng/ml; below 37 ng/ml; below 36 ng/ml; below 35 ng/ml; below 34 ng/ml; below 33 ng/ml; below 32 ng/ml; below 31 ng/ml; below 30 ng/mL; below 29 ng/ml; below 28 ng/ml; below 27 ng/ml; below 26 ng/ml; below 25 ng/ml; below 24 ng/ml; below 23 ng/ml; below 22 ng/ml; below 21 ng/ml; below 20 ng/mL; below 19 ng/ml; below 18 ng/ml; below 17 ng/ml; below 16 ng/ml; below 15 ng/mL; below 14 ng/ml; below 13 ng/ml; below 12 ng/ml; below 11 ng/ml; below 10 ng/ml; between 65 and 70 ng/ml; between 60 and 65 ng/ml; between 55 and 60 ng/ml; between 50 and 55 ng/ml; between 45 and 50 ng/ml; between 40 and 45 ng/ml; between 35 and 40 ng/ml; between 30 and 35 ng/ml; between 25 and 30 ng/ml; between 20 and 25 ng/ml; between 15 and 20 ng/ml; between 10 and 15 ng/ml; between 15 and 25 ng/ml; between 18 and 20 ng/ml; or between 10 and 30 ng/ml.

In certain embodiments, the maximum threshold may be a median concentration in a population. In other embodiments, the maximum threshold may be an average concentration in a population. The maximum threshold may, in some cases, be a concentration observed or exceeded in no more than 50% to 60% of a population; no more than 50% to 70% percent of a population; no more than 50% to 80% percent of a population; no more than 40% to 50% percent of a population; no more than 30% to 50% percent of a population; no more than 20% to 50% percent of a population; no more than 20% to 25% of a population; no more than 25% to 30% of a population; no more than 30% to 35% of a population; no more than 35% to 40% of a population; no more than 40% to 45% of a population; no more than 45% to 50% of a population; no more than 50% to 55% of a population; no more than 55% to 60% of a population; no more than 60% to 65% of a population; no more than 65% to 70% of a population; no more than 70% to 75% of a population; no more than 75% to 80% of a population; no more than 20% of a population; no more than 21% of a population; no more than 21% of a population; no more than 22% of a population; no more than 23% of a population; no more than 24% of a population; no more than 25% of a population; no more than 26% of a population; no more than 27% of a population; no more than 28% of a population; no more than 29% of a population; no more than 30% of a population; no more than 31% of a population; no more than 32% of a population; no more than 33% of a population; no more than 34% of a population; no more than 35% of a population; no more than 36% of a population; no more than 37% of a population; no more than 38% of a population; no more than 39% of a population; no more than 40% of a population; no more than 41% of a population; no more than 42% of a population; no more than 43% of a population; no more than 44% of a population; no more than 45% of a population; no more than 46% of a population; no more than 47% of a population; no more than 48% of a population; no more than 49% of a population; no more than 50% of a population; no more than 51% of a population; no more than 52% of a population; no more than 53% of a population; no more than 54% of a population; no more than 55% of a population; no more than 56% of a population; no more than 57% of a population; no more than 58% of a population; no more than 59% of a population; no more than 60% of a population; no more than 61% of a population; no more than 62% of a population; no more than 63% of a population; no more than 64% of a population; no more than 65% of a population; no more than 66% of a population; no more than 67% of a population; no more than 68% of a population; no more than 69% of a population; no more than 70% of a population; no more than 71% of a population; no more than 72% of a population; no more than 73% of a population; no more than 74% of a population; no more than 75% of a population; no more than 76% of a population; no more than 77% of a population; no more than 78% of a population; no more than 79% of a population; or no more than 80% of a population.

In some embodiments, the population may be the total population. In other embodiments, the population may be a subset of the total population. For example, in some cases, the population may be a group of patients (e.g., humans) belonging to, for example, an age group (e.g., patients that are 18 to 85 years old; 18 to 59 years old; 18 to 25 years old; 25 to 35 years old; 35 to 45 years old; 45 to 55 years old; 55 to 65 years old; 65 to 75 years old; at least 35 years old; at least 40 years old; at least 45 years old, at least 50 years old; at least 60 years old; or at least 65 years old), gender group (e.g., male or female), racial group (e.g., African, Asian, Caucasian, and/or Hispanic), and/or ethnic group.

In some embodiments, the population may be characterized by body mass index (BMI). For example, the population may be a group of patients having a BMI less than 18.5; between 18.5 and 24.9; between 25 and 29.9; or at least 30.

In some embodiments, the population may be a group of patients that have a medical precondition. For example, the population may be a group of patients that have had at least one heart attack. In another example, the population may be a group of patients that have had at least one stroke. In still another example, the population may be a group of patients with diabetes. In some embodiments, the population may be a group of patients that do not have a medical precondition. For example, the population may be a group of patients that have not had a heart attack. In another example, the population may be a group of patients that have not had a stroke. In still another example, the population may be a group of patients that do not have diabetes.

In some embodiments, the population may be a group of patients that have total cholesterol less than 200 mg/dL; between 200 and 239 mg/dL; or at least 240 mg/dL; LDL cholesterol less than 130 mg/dL; between 100 and 129 mg/dL; between 130 and 159 mg/dL; between 160 and 189 mg/dL; or at least 190 mg/dL; HDL cholesterol less than 40 mg/dL; between 41 mg/dL and 59 mg/dL; or at least 60 mg/dL; and/or triglycerides less than 150 mg/dL; between 150 and 199 mg/dL; between 200 and 499 mg/dL; or at least 500 mg/dL.

It should be understood that, in some embodiments, the population may be a group of patients having any combination of the population criteria described above.

In some embodiments, the blood concentration of other markers may be used in combination with the blood concentration of galectin-3 to predict the response of patients to a statin. For example, in some case, the levels of N-terminal pro B-type natriuretic peptide (NT-proBNP) may be combined with the levels of galectin-3. In some embodiments, the blood concentration of NT-proBNP may be determined to be above a minimum threshold, below a maximum threshold or within a target range defined by a minimum and a maximum threshold. The minimum threshold may be, for example, more than 200 pg/ml; between 200 and 1000 pg/ml; between 500 and 1000 pg/ml; or between 700 and 900 pg/ml. The maximum threshold may be, for example, below 1500 pg/ml; below 1200 pg/ml; below 1000 pg/ml; between 500 and 1500 pg/ml; between 1000 and 1500 pg/ml; or between 500 and 1000 pg/ml.

### Galectin-3 Detection:

Described herein are methods for predicting and/or monitoring the physiological response of a heart failure patient to treatment of heart failure with a statin compound by measuring the levels of markers such as galectin-3, optionally in combination with one or more other markers (e.g., B-type natriuretic peptide (BNP), NT-proBNP). Many methods for detecting of a protein of interest, with or without quantitation, are well known and can be used in the practice of the methods contemplated herein. Examples of such assays are described below and can include, for example, immunoassays, chromatographic methods, and mass spectroscopy. Such assays can be performed on any biological sample including, among others, blood, plasma, and serum. Accordingly, multiple assays can be used to detect galectin-3, and samples can be analyzed from one or more sources.

Markers can be detected or quantified in a sample with the help of one or more separation methods. For example, suitable separation methods may include a mass spectrometry method, such as electrospray ionization mass spectrometry (ESI-MS), ESI-MS/MS, ESMS/(MS)ⁿ (n is an integer greater than zero), matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), atmospheric pressure chemical ionization mass spectrometry (APC)-MS), APCI-MS/MS, APCI-(MS)ⁿ, or atmospheric pressure photoionization mass spectrometry (APPI-MS), APPI-MS/MS, and APPI-(MS)ⁿ. Other mass spectrometry methods may include, inter alia, quadrupole, fourier transform mass spectrometry (FTMS) and ion trap. Spectrometric techniques that can also be used include resonance spectroscopy and optical spectroscopy.

Other suitable separation methods include chemical extraction partitioning, column chromatography, ion exchange chromatography, hydrophobic (reverse phase) liquid chromatography, isoelectric focusing, one-dimensional polyacrylamide gel electrophoresis (PAGE), two-dimensional polyacrylamide gel electrophoresis (2D-PAGE), or other chromatographic techniques, such as thin-layer, gas or liquid chromatography, or any combination thereof. In one embodiment, the biological sample to be assayed may be fractionated prior to application of the separation method.

Markers may be detected or quantified by methods that do not require physical separation of the markers themselves. For example, nuclear magnetic resonance (NMR) spectroscopy may be used to resolve a profile of a marker from a complex mixture of molecules. An analogous use of NMR to classify tumors is disclosed in Hagberg (1998) NMR Biomed. 11:148-56, for example.

A marker in a sample also may be detected or quantified, for example, by combining the marker with a binding moiety capable of specifically binding the marker. The binding moiety may include, for example, a member of a ligand-receptor pair, i.e., a pair of molecules capable of having a specific binding interaction. The binding moiety may also include, for example, a member of a specific binding pair, such as antibody-antigen, enzyme-substrate, nucleic acid-nucleic acid, protein-nucleic acid, protein-protein, or other specific binding pairs known in the art. Binding proteins may be designed which have enhanced affinity for a target. Optionally, the binding moiety may be linked with a detectable label, such as an enzymatic, fluorescent, radioactive, phosphorescent or colored particle label. The labeled complex may be detected, e.g., visually or with the aid of a spectrophotometer or other detector, or may be quantified.

Galectin-3 levels can be quantitated by performing an immunoassay. A galectin-3 immunoassay involves contacting a sample from a subject to be tested with an appropriate antibody under conditions such that immunospecific binding can occur if galectin-3 is present, and detecting or measuring the amount of any immunospecific binding by the antibody. Any suitable immunoassay can be used, including, without limitation, competitive and non-competitive assay systems using techniques such as Western blots, radioimmunoassays, ELISA (enzyme linked immunosorbent assay), “sandwich” immunoassays, immunoprecipitation assays, immunodiffusion assays, agglutination assays, complement-fixation assays, immunoradiometric assays, fluorescent immunoassays and protein A immunoassays.

In a “sandwich” assay, two molecules (“binding moieties”) such as monoclonal antibodies that specifically bind to non-overlapping sites (epitopes) on galectin-3 are used. Typically, one binding moiety is immobilized on a solid surface where it binds with and captures galectin-3. This first binding moiety is therefore also referred to as the capture binding moiety. A second binding moiety is detectably labeled, for example, with a fluorophore, enzyme, or colored particle, such that binding of the second binding moiety to the galectin-3-complex indicates that galectin-3 has been captured. The intensity of the signal is proportional to the concentration of Gal-3 in the sample. The second binding moiety is therefore also referred to as the detection binding moiety or label binding moiety. A binding moiety can be any type of molecule, as long as it specifically binds to a portion of the N-terminus of galectin-3. In a preferred embodiment, the binding moieties used are monoclonal anti-galectin-3 antibodies, i.e., monoclonals raised against or otherwise selected to bind to separate portions of galectin-3.

Such assay procedures can be referred to as two-site immunometric assay methods, “sandwich” methods or (when antibodies are the binders) “sandwich immunoassays.” As is known in the art, the capture and detection antibodies can be contacted with the test sample simultaneously or sequentially. Sequential methods can be accomplished by incubating the capture antibody with the sample, and adding the labeled detection antibody at a predetermined time thereafter (sometimes referred to as the “forward” method). Alternatively, the labeled detection antibody can be incubated with the sample first and then the sample can be exposed to the capture antibody (sometimes referred to as the “reverse” method). After any necessary incubation(s), which may be of short duration, to complete the assay, the label is measured. Such assays may be implemented in many specific formats known to those of skill in the art, including through use of various high throughput clinical laboratory analyzers or with a point of care or home testing device.

In one embodiment, a lateral flow device may be used in the sandwich format wherein the presence of galectin-3 above a baseline sensitivity level in a biological sample will permit formation of a sandwich interaction upstream of or at the capture zone in the lateral flow assay. See: for example, U.S. Pat. No. 6,485,982. The capture zone may contain capture binding moieties such as antibody molecules, suitable for capturing galectin-3, or immobilized avidin or the like for capture of a biotinylated complex. See: for example, U.S. Pat. No. 6,319,676. The device may also incorporate a luminescent label suitable for capture in the capture zone, the concentration of galectin 3 being proportional to the intensity of the signal at the capture site. Suitable labels include fluorescent labels immobilized on polystyrene microspheres. Colored particles also may be used.

Other assay formats that may be used in the methods of the invention include, but are not limited to, flow-through devices. See for example, U.S. Pat. No. 4,632,901. In a flow-through assay, one binding moiety (for example, an antibody) is immobilized to a defined area on a membrane surface. This membrane is then overlaid on an absorbent layer that acts as a reservoir to pump sample volume through the device. Following immobilization, the remaining protein-binding sites on the membrane are blocked to minimize non-specific interactions. In operation, a biological sample is added to the membrane and filters through the matrix, allowing any analyte specific to the antibody in the sample to bind to the immobilized antibody. In a second step, a labeled secondary antibody may be added or released that reacts with captured marker to complete the sandwich. Alternatively, the secondary antibody can be mixed with the sample and added in a single step. If galectin-3 is present, a colored spot develops on the surface of the membrane.

The most common enzyme immunoassay is the “Enzyme-Linked Immunosorbent Assay (ELISA).” ELISA is a technique for detecting and measuring the concentration of an antigen using a labeled (e.g., enzyme linked) form of the antibody. There are different forms of ELISA, which are well known to those skilled in the art. Standard ELISA techniques are described in “Methods in Immunodiagnosis”, 2nd Edition, Rose and Bigazzi, eds. John Wiley & Sons, 1980; Campbell et al., “Methods and Immunology”, W. A. Benjamin, Inc., 1964; and Oellerich, M. (1984), J. Clin. Chem. Clin. Biochem. 22:895-904. A preferred enzyme-linked immunosorbant assay kit (ELISA) for detecting galectin-3 is commercially available (BG Medicine, Waltham, Mass.).

In a “sandwich ELISA,” an antibody (e.g., anti-galectin-3) is linked to a solid phase (i.e., a microtiter plate) and exposed to a biological sample containing antigen (e.g., galectin-3). The solid phase is then washed to remove unbound antigen. A labeled antibody (e.g., enzyme linked) is then bound to the bound-antigen (if present) forming an antibody-antigen-antibody sandwich. Examples of enzymes that can be linked to the antibody are alkaline phosphatase, horseradish peroxidase, luciferase, urease, and β-galactosidase. The enzyme linked antibody reacts with a substrate to generate a colored reaction product that can be measured. Any of the immunoassays described herein suitable for use with the kits and methods of the present invention can also use any binding moiety in the place of an antibody.

A detailed review of immunological assay design, theory and protocols can be found in numerous texts in the art, including Butt, W. R., Practical Immunology, ed. Marcel Dekker, New York (1984) and Harlow et al. Antibodies, A Laboratory Approach, ed. Cold Spring Harbor Laboratory (1988).

In general, immunoassay design considerations include preparation of antibodies (e.g., monoclonal or polyclonal antibodies) having sufficiently high binding specificity for the target to form a complex that can be distinguished reliably from products of nonspecific interactions. As used herein, the term “antibody” is understood to mean binding proteins, for example, antibodies or other proteins comprising an immunoglobulin variable region-like binding domain, having the appropriate binding affinities and specificities for the target. The higher the antibody binding specificity, the lower the target concentration that can be detected. As used herein, the terms “specific binding” or “binding specifically” are understood to mean that the binding moiety, for example, a binding protein, has a binding affinity for the target of greater than about 10⁵ M⁻¹, more preferably greater than about 10⁷ M⁻¹.

Antibodies to an isolated target marker which are useful in assays for detecting heart failure in an individual may be generated using standard immunological procedures well known and described in the art. See, for example Practical Immunology, supra. Briefly, an isolated marker is used to raise antibodies in a xenogeneic host, such as a mouse, goat or other suitable mammal. The marker is combined with a suitable adjuvant capable of enhancing antibody production in the host, and is injected into the host, for example, by intraperitoneal administration. Any adjuvant suitable for stimulating the host's immune response may be used. A commonly used adjuvant is Freund's complete adjuvant (an emulsion comprising killed and dried microbial cells and available from, for example, Calbiochem Corp., San Diego, or Gibco, Grand Island, N.Y.). Where multiple antigen injections are desired, the subsequent injections may comprise the antigen in combination with an incomplete adjuvant (e.g., cell-free emulsion). Polyclonal antibodies may be isolated from the antibody-producing host by extracting serum containing antibodies to the protein of interest. Monoclonal antibodies may be produced by isolating host cells that produce the desired antibody, fusing these cells with myeloma cells using standard procedures known in the immunology art, and screening for hybrid cells (hybridomas) that react specifically with the target and have the desired binding affinity.

Exemplary epitopes from the N-terminus of galectin-3 include, but are not limited to, MADNFSLHDALS; MADNFSLHDALSGS; WGNQPAGAGG; YPGAPGAYPGAPAPGV; GNPNPQGWPGA; YPSSGQPSATGA; YPGQAPPGAYPGQAPPGA; YPGAPAPGVYPGPPSGPGA; and YPSSGQPSATGA. Other galectin-3 epitopes, including non-linear epitopes, can also be used as targets for detection by an anti-galectin-3 antibody. Exemplary antibodies are discussed in U.S. Application No. 61/109,366, the entire contents of which are incorporated herein by reference.

Antibody binding domains also may be produced biosynthetically and the amino acid sequence of the binding domain manipulated to enhance binding affinity with a preferred epitope on the target. Specific antibody methodologies are well understood and described in the literature. A more detailed description of their preparation can be found, for example, in Practical Immunology, (supra).

In addition, genetically engineered biosynthetic antibody binding sites, also known in the art as BABS or sFv's, may be used to determine if a sample contains a marker. Methods for making and using BABS comprising (i) non-covalently associated or disulfide bonded synthetic V_{H} and V_{L} dimers, (ii) covalently linked V_{H}-V_{L} single chain binding sites, (iii) individual V_{H} or V_{L} domains, or (iv) single chain antibody binding sites are disclosed, for example, in U.S. Pat. Nos. 5,091,513; 5,132,405; 4,704,692; and 4,946,778. Furthermore, BABS having requisite specificity for the marker can be derived by phage antibody cloning from combinatorial gene libraries (see, for example, Clackson et al. Nature 352: 624-628 (1991)). Briefly, phages, each expressing on their coat surfaces BABS having immunoglobulin variable regions encoded by variable region gene sequences derived from mice pre-immunized with an isolated marker, or a fragment thereof, are screened for binding activity against the immobilized marker. Phages which bind to the immobilized marker are harvested and the gene encoding the BABS is sequenced. The resulting nucleic acid sequences encoding the BABS of interest then may be expressed in conventional expression systems to produce the BABS protein.

Multimarker analysis can be used to improve the accuracy of diagnosis and monitoring. For example, blood concentrations of galectin-3 (Gal-3) and brain natriuretic peptide (BNP) can be used to diagnose heart failure and to predict the long-term outcome of heart failure (van Kimmenade et al., J. Am. Coll. Cardiol., 48:1217-24 (2006); Sharma et al., Circulation, 110:3121-28 (2004); Lok et al., Eur. Heart J., 28:141, Abstract 1035 (2007)). BNP and its cleavage equivalent amino-terminal proBNP(NT-proBNP) are elevated in heart muscle and in blood during heart failure as a result of high filling pressures of heart chambers and the stretch of cardiac muscle fibers. Other secondary markers that could be used to diagnose heart failure may include non-polypeptidic cardiac markers such as sphingolipid, sphingosine, sphingosine-1-phosphate, dihydrosphingosine and sphingosylphosphorylcholine (see U.S. Pat. No. 6,534,322). When measuring the levels of the above markers, corrections for age and gender may be incorporated to improve the accuracy of diagnosis.

### Treatment Methods

Patients whose galectin-3 levels identify them as candidates for statin treatment can be treated by repeated administration of a statin. Rosuvastatin, for example, can be administered in accordance with the manufacturer's instructions, or those of a prescribing physician. Generally, rosuvastatin is administered orally at a dose of between 5 and 40 mg once per day. Atorvastatin is generally administered at a dose of between 10 and 80 mg once per day; the same is true for pravastatin. Fluvastatin is generally administered at a dose of between 20 and 80 mg per day, either as a single dose or as two doses (e.g. 40 mg+40 mg). Lovastatin is generally administered at 10-40 mg/day, as a single dose or as two divided doses. Pitavastatin is generally administered at 1-4 mg/day, whereas simvastatin is generally administered at 5-80 mg/day.

Treatment with a statin is optionally combined with one or more other treatments for heart failure. For example, a patient may also be treated with: a diuretic, such as furosemide, bumetanide, hydrochlorothiazide, spironolactone, eplerenone, triamterene, torsemide, or metolazone; an inotrope, such as dobutamine, milrinone, or digoxin; a beta-blocker, such as carvediol or metoprolol; and/or a natriuretic peptide, such as BNP. Treatments can also include a vasodilator, such as: an angiotensin-converting enzyme (ACE) inhibitor (e.g. captopril, enalapril, lisinopril, benazepril, quinapril, fosinopril, or ramipril); an angiotensin II receptor blocker, such as candesartan, irbesartan, olmesartan, losartan, valsartan, telmisartan, or eprosartan; a nitrate, such as isosorbide mononitrate or isosorbide dinitrate; and/or hydralazine. Other forms of medical intervention, such as angioplasty, implantation of a pacemaker, or other surgery can also be performed in appropriate cases.

Galectin-3 levels and/or other biomarkers (such as BNP) can be measured in a patient taking a statin and can be compared to a previous galectin-3 concentration measured in the patient. An increase or decrease in galectin-3 concentration relative to one or more previous galectin-3 concentrations in the patient may be an indication that the patient is responding or not responding to statin therapy. Marker levels can be monitored over time, such as in samples obtained from a patient at annual, semi-annual, bimonthly, monthly, triweekly, biweekly, weekly, daily, or at variable intervals.

Treatment with a statin may be modified if the patient is determined to be not responding to statin therapy. For example, the statin dosage amount may be increased or the frequency of administration may be increased until the patient's level of galectin-3 is reduced to an acceptable level.

The invention is further illustrated by the following example. The example is provided for illustrative purposes only, and is not to be construed as limiting the scope or content of the invention in any way.

### EXAMPLES

### Example 1

### Galectin-3 Levels in Heart Failure Patients Receiving Statin Therapy

The concentration of the protein galectin-3 was measured using the BG Medicine, Inc. Galectin-3 ELISA assay (BG Medicine Inc., Waltham, Mass.), according to the manufacturer's instructions, in de-identified, banked plasma specimens that were collected during the conduct of a prospective study including acute decompensated HF subjects. The study was a prospective observational cohort study conducted at multiple US centers that enrolled and followed for at least one year consenting subjects aged 18 years or older who presented with dyspnea to the emergency department. Major exclusion criteria included overt causes of dyspnea including trauma, pneumothorax, or upper airway obstruction, and diagnosis of acute coronary syndrome. An expert panel of physicians adjudicated a diagnosis of acute decompensated heart failure in a subset of the enrolled subjects. Blood plasma samples were collected at baseline (upon enrollment) from subjects participating in the study and were banked, and were available for analysis. At the time of enrollment in the study a blood sample was collected from the study subjects into tubes containing EDTA. The blood was processed and blood plasma was subsequently frozen and stored at 70° C. or colder. Approximately 72.1% of the heart failure subjects were male. The mean (SD) age at baseline of these male heart failure subjects was 60.9 (12.7) years. The mean (SD) age at baseline of female heart failure subjects included was 63.5 (15.0) years. Each subject was followed for at least one year and death or re-hospitalization for heart failure were recorded if occurring during this follow-up period.

Of the heart failure patients in the study, 38 heart failure patients were identified who were on a statin medication at the time of blood collection. The statin medications included atorvastatin, simvastatin, and rosuvastatin. The concentration of galectin-3 was determined using the BG Medicine, Inc. Galectin-3 Assay in the baseline blood plasma samples of these patients. Fourteen (14) of these 38 heart failure patients died during the follow-up period, and the other 24 were still alive as of the end of the follow-up period.

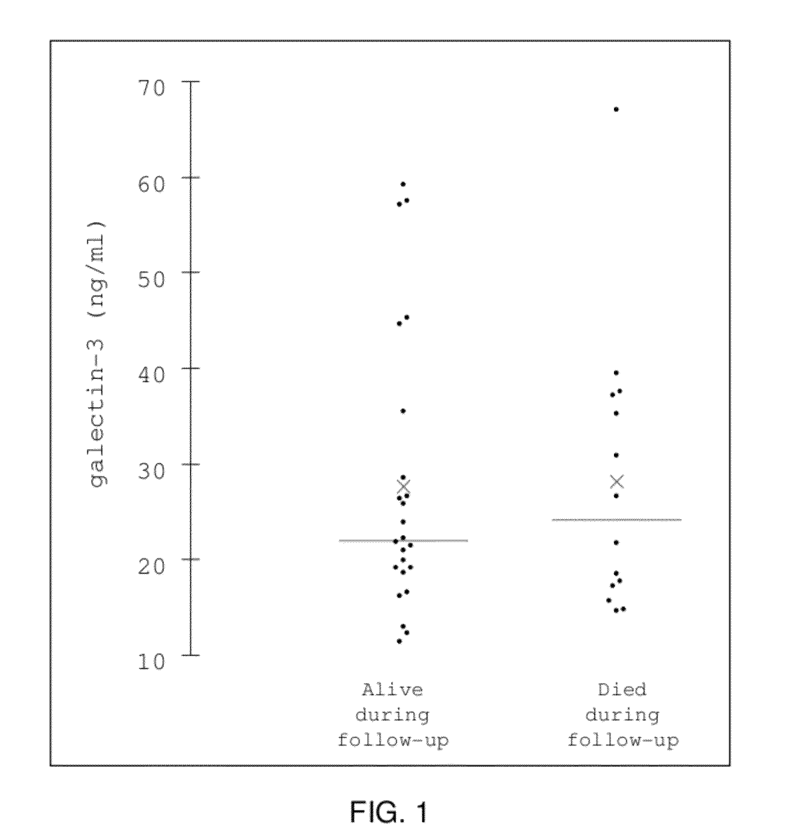

As shown in FIG. 1, it was observed that heart failure patients who were receiving statin therapy and who died during the follow-up period had a higher baseline galectin-3 concentration in plasma than patients who were receiving statin therapy and who did not die during the follow-up period.

### Example 2

### Galectin-3 and Statin Therapy

### Methods

### Study Population

A clinical trial enrolled patients with chronic heart failure (HF) (New York Heart Association functional class II, III or IV) and with left ventricular ejection fraction less than or equal to 40% (less than or equal to 35% for patients in New York Heart Association functional class II). The design and primary results of the trial are reported in published literature [J. Kjekshus et al., “Rosuvastatin in Older Patients with Systolic Heart Failure.” N Engl J. Med. 2007 Nov. 29; 357(22):2248-61]. Patients were randomly assigned to receive 10 milligrams of rosuvastatin or placebo per day. Of the patients in the trial, a subset of 1,462 subjects also participated in a sub-study in which blood was collected at baseline, defined as the time of enrollment in the trial. Baseline galectin-3 levels were evaluated in these 1,46 s subjects.

### Blood Sampling

Blood plasma samples were drawn at baseline from participants in a non-fasting state. All samples were stored at −80° C. Plasma galectin-3 levels were determined on these plasma specimens using a commercial enzyme-linked immunosorbent assay (BGM Galectin-3 Assay; BG Medicine, Waltham, Mass.) according to the manufacturer's instructions.

### Laboratory Analysis

Determination of galectin-3 concentration in serum samples was assessed using ELISA kits (BGM Galectin-3 Assay; BG Medicine Inc., Waltham, Mass., USA). The assay sensitivity (lowest concentration different from zero) was 0.96 ng/mL. Intra- and inter-assay variations were less than 8% and 10% respectively.

### Statistical Analyses

For the main objective of investigating the effects of rosuvastatin treatment according to baseline galectin-3 level, galectin-3 categories were defined based on the median baseline value across all 1,462 subjects for whom a galectin-3 measurement was available. For each of six endpoints, a formal interaction test was conducted within a Cox proportional hazards model comprising treatment group (binary variable), galectin-3 category (dichotomous variable), and the interaction term. Subsequently, Cox proportional hazards models were used to estimate the hazard ratios (HRs) and 95% confidence intervals (CIs) comparing rosuvastatin and placebo treatments by galectin-3 categories. Fully adjusted models included the following 11 covariates, all evaluated at baseline: age (per year), gender, LVEF (per unit), NYHA class (due to the small number of subjects with class IV, class III and IV were combined), body mass index (BMI) (per unit), diabetes mellitus (yes/no), intermittent claudication (yes/no), heart rate (per unit), estimated glomerular filtration rate (eGFR) (per unit), the ratio of apolipoprotein B to apolipoprotein A1, and N-terminal B-type natriuretic peptide (NT-proBNP) (the variable NT-proBNP was logarithmically transformed using base e). Incident event rates were analyzed using Poisson regression. Baseline characteristics were compared by galectin-3 category using Student's t-test for variables expressed as means and standard deviation. All P-values are two two-tailed. All analyses were performed with SAS software, version 9.1 (SAS Institute), or R software, version 2.

### Results

### Baseline Galectin-3 Predicts Response to Statin Therapy

Of the 5,011 patients enrolled in the trial, 1,462 subjects had a baseline plasma specimen available for measurement of galectin-3. The median baseline galectin-3 value across these 1,462 subjects was 19.0 ng/mL. Baseline galectin-3 levels were similar among subjects randomized to rosuvastatin (N=737; median, 19.1 ng/mL; interquartile range (IQR), 15.5-23.6 ng/mL) and to placebo (N=725; median, 18.9 ng/mL; IQR, 15.6-23.9 ng/mL; P=0.85 compared to rosuvastatin group). The 1,462 subjects were categorized by the median baseline galectin-3 value of 19.0 ng/mL into two categories, namely those with baseline galectin-3 level≦19.0 ng/mL, and those with baseline galectin level>19.0 ng/mL.

There was a significant interaction between baseline galectin-3 category, defined by the 19.0 ng/mL value, and the effect of rosuvastatin on the primary endpoint of the earlier of cardiovascular death, non-fatal myocardial infarction or non-fatal stroke (P=0.036 for interaction). Among subjects with baseline galectin-3 less than or equal to the median value of 19.0 ng/mL (N=734), rosuvastatin treatment was associated with a decreased risk of the primary endpoint compared to placebo (hazard ratio (HR) adjusted for all 11 clinical and biochemical covariates: 0.65; 95% confidence interval (CI), 0.46-0.92). In this low baseline galectin-3 group, the rate of primary events among subjects randomized to rosuvastatin was 7.8 events per 100 patient-years, compared to 11.2 events per 100 patient-years of follow-up in the placebo group, representing a 30.4% difference (P=0.019 for comparison of rates). In contrast, among subjects with baseline galectin-3 levels>19.0 ng/mL (N=728), rosuvastatin treatment was not associated with benefit compared to placebo (adjusted HR, 1.07; 95% CI (0.79-1.45); P=0.66), and event rates were comparable (15.1 events per 100 patient-years in the rosuvastatin group, compared to 14.2 events per 100 patient-years in the placebo group, P=0.61). Table 1 summarizes these results.

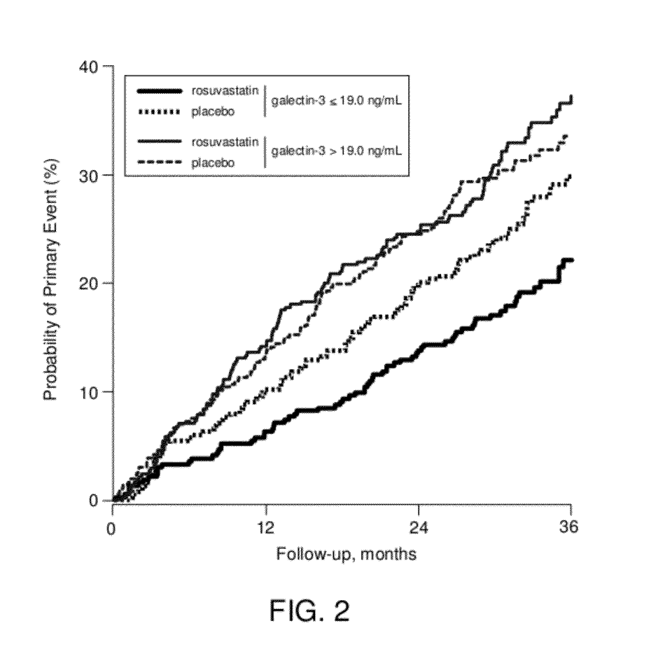

Kaplan Meier probability estimates by treatment group and by baseline galectin-3 level are shown for the primary endpoint in FIG. 2 by galectin-3 category (above and at or below median level, 19.0 ng/mL). Rosuvastatin treatment was associated with a significantly decreased risk of the primary endpoint compared to placebo (hazard ratio adjusted for 11 clinical and biochemical covariates: 0.65; 95% confidence interval, 0.46-0.92; P=0.014) among subjects with baseline galectin-3 less than or equal to the median value of 19.0 ng/mL (N=734).

TABLE 1
Rate of subjects experiencing the primary outcome
by baseline galectin-3 category and treatment.

| | | Placebo | Rosuvastatin | |
|---|---|---|---|---|
| | | rate (per 100 | rate (per 100 | |
| | number of | person-years | person-years | |
| | subjects | of follow-up) | of follow-up) | p value |
| Galectin-3 ≦ | 734 | 11.2 | 7.8 | 0.019 |
| 19.0 ng/mL | | | | |
| Galectin-3 > | 728 | 14.2 | 15.1 | 0.61 |
| 19.0 ng/mL | | | | |

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

What is claimed is:

1. A method of selecting a therapy for a human, the method comprising measuring a galectin-3 blood concentration in a sample from the human, thereby to determine the presence or absence of a galectin-3 blood concentration indicative of responsiveness to an inhibitor (statin) of 3-hydroxy-3-methylglutaryl-coenzyme A reductase.

2. The method of claim 1, wherein the sample comprises blood, serum or plasma.

3. The method of claim 1, further comprising repeatedly administering the statin to the human.

4. A method of treating a human comprising repeatedly administering a 3-hydroxy-3-methylglutaryl-coenzyme A reductase inhibitor (statin) to a human having a determined galectin-3 blood concentration indicative of a survival-enhancing response to the statin.

5. The method of claim 4 comprising the additional step of monitoring the human's galectin-3 blood concentration over the course of the therapy.

6. The method of claim 4, wherein the statin is administered in an amount sufficient to inhibit progression or development of heart failure.

7. The method of claim 4, wherein the statin is administered in a survival-enhancing amount.

8. The method of claim 4, wherein the statin is administered in a myocardial infarction risk-reducing amount.

9. The method of claim 4, wherein the statin is administered in a stroke risk-reducing amount.

10. The method of claim 4, wherein the statin is selected from the group consisting of atorvastatin, cerivastatin, fluvastatin, lovastatin, mevastatin, pitavastatin, pravastatin, rosuvastatin, and simvastatin.

11. The method of claim 10, wherein the statin is rosuvastatin.

12. The method of claim 11, wherein the statin is administered at a dose of between 5 and 40 mg/day.

13. The method of claim 10, wherein the statin is atorvastatin.

14. The method of claim 13, wherein the statin is administered at a dose of between 10 and 80 mg/day.

15. The method of claim 4, wherein the human has a galectin-3 blood concentration determined to be within a target range.

16. The method of claim 4, wherein the human has a galectin-3 blood concentration determined to be below a maximum threshold.

17. The method of claim 16, wherein the maximum threshold is below 70 ng/ml.

18. The method of claim 16, wherein the maximum threshold is below 60 ng/ml.

19. The method of claim 16, wherein the maximum threshold is below 40 ng/ml.

20. The method of claim 16, wherein the maximum threshold is below 30 ng/ml.

21. The method of claim 16, wherein the maximum threshold is below 20 ng/ml.

22. The method of claim 16, wherein the maximum threshold is below 15 ng/ml.

23. The method of claim 16, wherein the maximum threshold is between 30 and 40 ng/ml.

24. The method of claim 16, wherein the maximum threshold is between 25 and 30 ng/ml.

25. The method of claim 16, wherein the maximum threshold is between 20 and 25 ng/ml.

26. The method of claim 16, wherein the maximum threshold is between 15 and 20 ng/ml.

27. The method of claim 16, wherein the maximum threshold is between 10 and 15 ng/ml.

28. The method of claim 16, wherein the maximum threshold is between 15 and 25 ng/ml.

29. The method of claim 16, wherein the maximum threshold is between 10 and 30 ng/ml.

30. The method of claim 16, wherein the maximum threshold is between 18 and 20 ng/ml.

31. The method of claim 16, wherein the maximum threshold is a median concentration in a population.

32. The method of claim 16, wherein the maximum threshold is an average concentration in a population.

33. The method of claim 16, wherein the maximum threshold is a concentration observed or exceeded in no more than 50% to 60% percent of a population.

34. The method of claim 16, wherein the maximum threshold is a concentration observed or exceeded in no more than 50% to 70% of a population.

35. The method of claim 16, wherein the maximum threshold is a concentration observed or exceeded in no more than 50% to 80% of a population.

36. The method of claim 16, wherein the maximum threshold is a concentration observed or exceeded in no more than 40% to 50% of a population.

37. The method of claim 16, wherein the maximum threshold is a concentration observed or exceeded in no more than 30% to 50% of a population.

38. The method of claim 16, wherein the maximum threshold is a concentration observed or exceeded in no more than 20% to 50% of a population.
